(19) United States
(12) Patent Application Publication
(10) Pub. No.: US 2008/0139668 A1
(43) Pub. Date: Jun. 12, 2008

(54) PHARMACEUTICAL COMPOSITION FOR OBOVATOL FOR THE PREVENTION AND TREATMENT OF RESTENOSIS

(75) Inventors: Byoung Mog Kwon, Daejeon (KR); Yeo-Pyo Yun, Chungcheongbuk-Do (KR); Yong Lim, Daejeon (KR); Dong-Woon Kim, Chungcheongbuk-Do (KR); Jin-Sook Kwon, Chungcheongbuk-Do (KR); Seung-ho Lee, Chungbuk (KR); Jin-Tae Hong, Chungcheongbuk-Do (KR)

Correspondence Address: LUCAS & MERCANTI, LLP, 475 PARK AVENUE SOUTH, 15TH FLOOR, NEW YORK, NY, 10016, US

(73) Assignees: Korea Research Institute of Bioscience and Biotechnology, Daejeon (KR); Chungbuk National University Industry-Academic Cooperation Foundation, Chungcheongbuk-do (KR)

(21) Appl. No.: 11/951,710

(22) Filed: Dec. 6, 2007

(30) Foreign Application Priority Data
Dec. 12, 2006 (KR) 10-2006-0126449

(51) Int. Cl. A61K 31/05; A61P 9/00

(52) U.S. Cl. 514/736

(57) ABSTRACT

Disclosed herein is a pharmaceutical composition for the prevention and treatment of restenosis following a blood vessel injury procedure, comprising obovatol as an active ingredient.

### BACKGROUND OF THE INVENTION

1. Field of the Invention

The present invention relates to PHARMACEUTICAL COMPOSITION FOR OBOVATOL FOR THE PREVENTION AND TREATMENT OF RESTENOSIS. More particularly, the present invention relates to a pharmaceutical composition useful in the prevention and treatment of restenosis following a stenting procedure, comprising obovatol as an active ingredient and a pharmaceutically acceptable carrier.

2. Description of the Related Art

Cardiovascular diseases, such as cardiac failure, coronary artery disease, hypertensive heart disease, arrhythmia, congenital heart defects, myocardial infarction, angina pectoris, apoplexy, and peripheral vascular (arterial) disease, afflict persons of various ages and, unless treated appropriately, leave serious sequelae or lead to death. Particularly, the morbidity of coronary artery diseases has recently sharply increased with the westernization of the Korean diet. Thus, many attempts have been made to develop effective therapy for coronary artery diseases.

Examples of the therapies for coronary artery diseases developed thus far include chemical therapy, gene therapy, and revascularization therapy, such as non-surgical percutaneous transluminal coronary angioplasty and stenting (PTCA) and surgical coronary artery bypass graft (CABG)

Thanks to advantages in that it is less invasive and more cost effective, percutaneous transluminal coronary angioplasty and stenting (PTCA) has become a widespread technique for the treatment of coronary artery disease. However, the utility of percutaneous transluminal coronary angioplasty (PTCA) is limited by a high incidence of restenosis following the procedure (‘post-PTCA restenosis’), which occurs in as many as 40% of cases within 3 to 6 months of the procedure (Ryan et al., J. Am. Coll. Cardiol., 22. 2033-2054, 1993).

It is well documented that chronic or acute injury (such as from a balloon used in PTCA) to the arterial wall induces the expression of a variety of growth factors and inflammatory cytokines that stimulate smooth muscle cell (SMC) proliferation and migration from the media into the intima, with the synthesis and secretion of extracellular matrix (ECM), resulting in neointimal formation and eventual restenosis (Godfried et al, Am. Heart J., 129, 203-210, 1995).

While not proliferating under normal conditions, vascular smooth muscle cells are induced to differentiation, migration and proliferation by signals transduced through multiple stages when the medial endothelial cells are injured by, for example, stenting. The removal of cell proliferation inhibitors and the activation of cell proliferation-stimulating factors, which occur upon the injury of normal endothelial cells, may explain the mechanism of vascular smooth muscle cell proliferation. For the mechanism, the transduction of proliferation-stimulating signals through receptors on vascular smooth muscle cell and the change in cell cycle induced by the proliferation-stimulating signals transferred to the nuclei of vascular smooth muscle cells are also responsible. Normal endothelial cells secrete factors inhibiting the proliferation of vascular smooth muscle cells. It is known that when endothelial cells are injured, the secretion is restrained while the proliferation of vascular smooth muscle cells is induced by platelet-derived growth factors, secreted from activated platelets and by various cytokines present in plasma.

Various methods for preventing restenosis following stenting (“post-PTCA restenosis”) have been studied. For example, Herdeg et al. reported that taxol is effective for the prevention of restenosis following angioplasty (Herdeg et al., Zeischrift fur Kardiologie, 89, 390-397, 1999). Korean Patent No. 478671 discloses a pharmaceutical composition for the prevention and treatment of restenosis, comprising clotrimazole as an active ingredient. Also, disclosed are a composition for the prevention and treatment of restenosis comprising 3′-deoxyadenosine in Korean Patent No. 516026, an anti-restenosis composition comprising an Rho kinase inhibitor in Korean Patent Laid-Open Publication No. 2001-110793, and antithrombin for the prevention and therapy of vasculoproliferative disorders, such as restenosis, in-stent restenosis and pulmonary hypertension, in Korean Patent Laid-Open Publication No. 2003-46314. Korean Patent Laid-Open Publication No. 2005-23249 provides medicament for prophylactic and/or therapeutic treatment of a vascular disease such as vascular restenosis and/or reocclusion after percutaneous transluminal coronary angioplasty using an intravascular stent, which comprises as an active ingredient a retinoid or an agent for controlling the action of retinoids. Another pharmaceutical composition for the prevention and treatment of restenosis comprising curcumin is described in Korean Patent Laid-Open Publication No. 2005-43183.

However, the above-mentioned anti-restenosis agents suffer from the disadvantages of wound healing suppression, vascular injury, hepatotoxicity, nephrotoxicity, and hemorrhage increase by platelet aggregation inhibition. Accordingly, active study has been conducted into the development of anti-restenosis agents from various natural materials confirmed to be safe to humans. No outstanding results have been reported thus far.

Therefore, there is a need for natural materials that can effectively prevent restenosis and are safe for the human body.

Of the natural materials, obovatol was found to be able to inhibit the proliferation of vascular smooth muscle cells, as a result of the study of the present inventors. A pharmaceutical composition comprising obovatol was already disclosed in Korean Patent Publication No. 2006-115454, issued to the present inventors, but is directed to the prevention and treatment of anxiety.

Leading to the present invention, intensive and thorough research into a safe anti-restenosis material, conducted by the present inventors, resulted in the finding that naturally occurring obovatol can inhibit the proliferation of vascular smooth muscle cells, thus being useful in the prevention of restenosis following stenting.

### SUMMARY OF THE INVENTION

Accordingly, the present invention has been made keeping in mind the above problems occurring in the prior art, and an object of the present invention is to provide an agent effective in the prevention and treatment of restenosis and safe for the human body.

In order to accomplish the above object, the present invention provides a pharmaceutical composition for the prevention and treatment of restenosis following a blood vessel injury procedure, comprising obovatol as an active ingredient.

The blood vessel injury procedure includes percutaneous transluminal coronary angioplasty, balloon angioplasty, stent insertion, coronary artery bypass graft surgery, and/or arteriovenous anastomosis.

The pharmaceutical composition is in the dosage form of a capsule, a liquid, an injection, a soft capsule, a granule or a tablet.

The obovatol useful in the present invention is derived from an extract from leaves of Magnolia obovata. The compound may be isolated from the leaves of Magnolia obovata as will be described below, or may be synthesized according to a method well known in the art.

### BREIF DESCRIPTION OF THE DRAWINGS

The above and other objects, features and other advantages of the present invention will be more clearly understood from the following detailed description taken in conjunction with the accompanying drawings, in which:

FIG. 1 shows the injured carotid arteries of control rat in cineangiography (×100),

FIG. 2 shows the injured carotid arteries of obovatol-treated rat in cineangiography (×100)

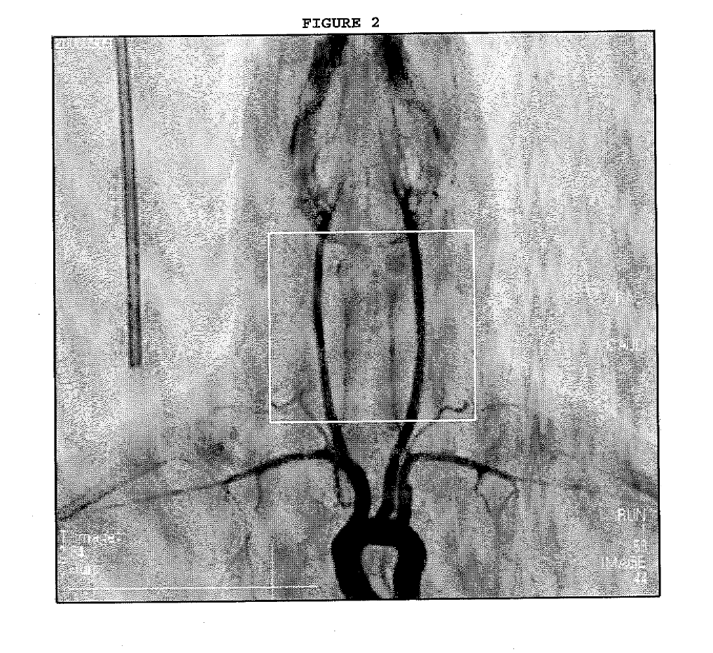

FIG. 3 shows the injured carotid arteries of control rat in cineangiography (×400),

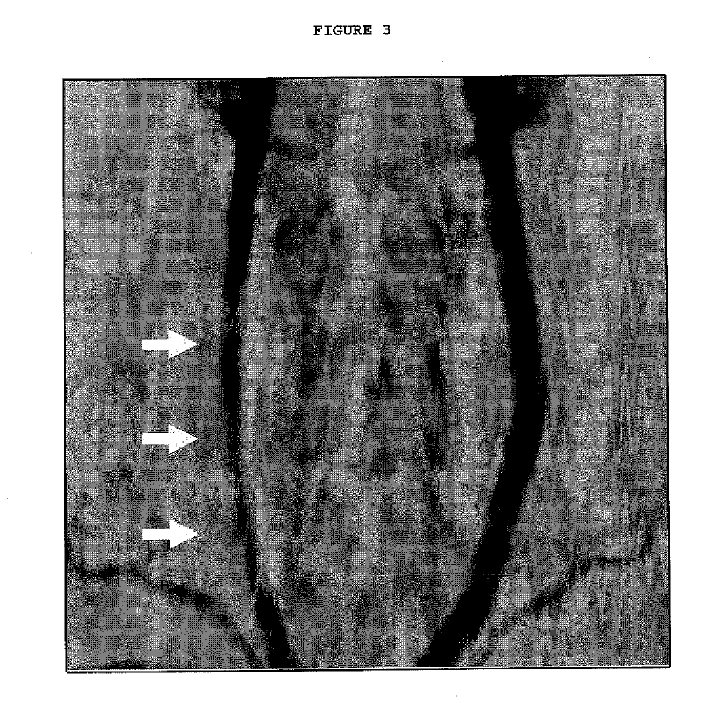

FIG. 4 shows the injured carotid arteries of obovatol-treated rat in cineangiography (×400),

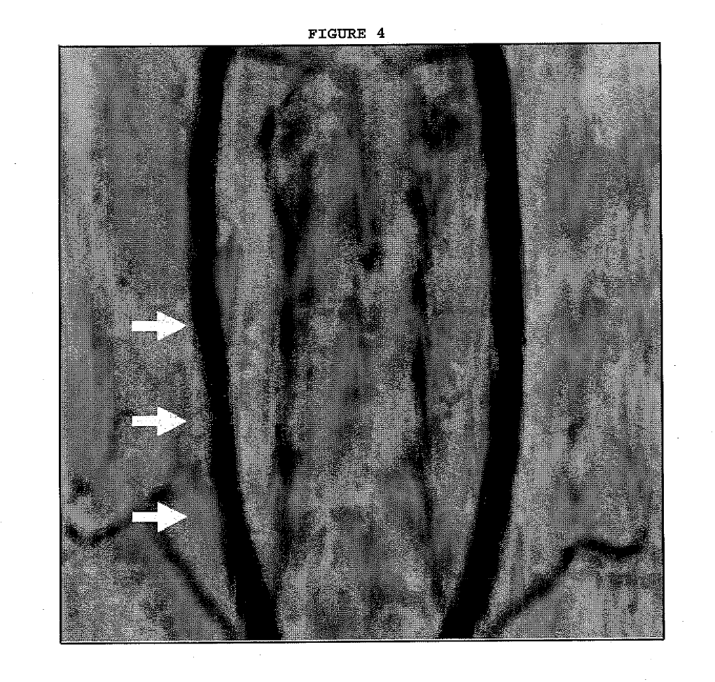

FIG. 5 is photograph showing cross sections of the injured carotid arteries of a control rat (×40),

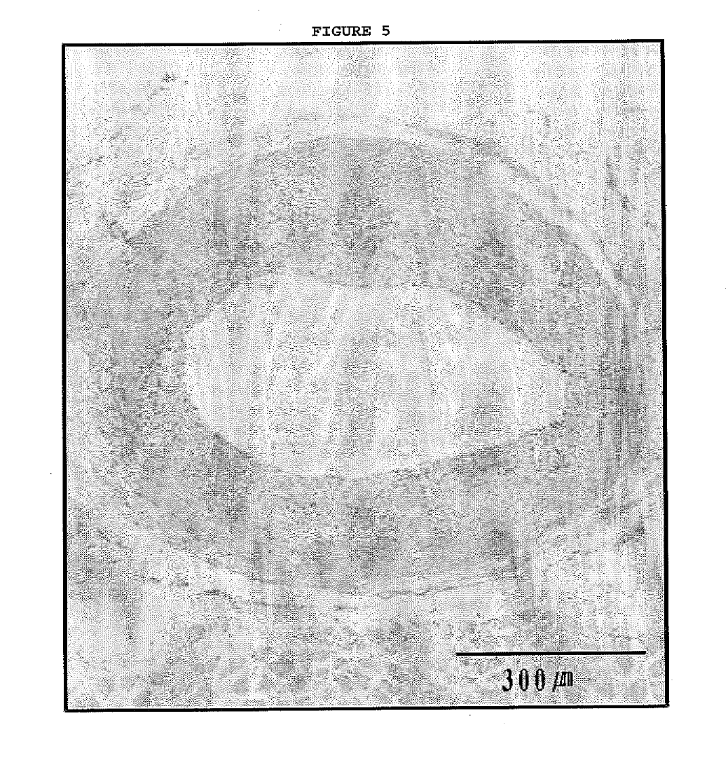

FIG. 6 is photograph showing cross sections of the injured carotid arteries of an obovatol-treated group, respectively (×40)

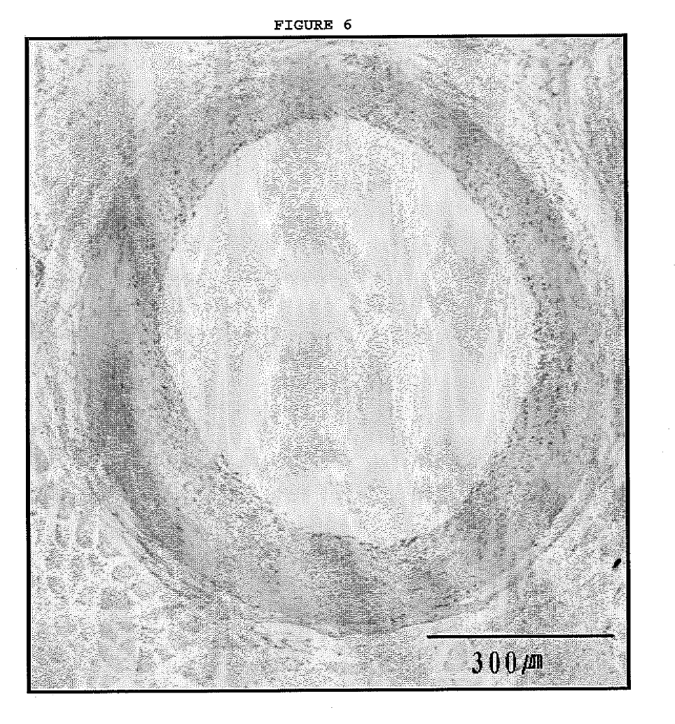

FIG. 7 is photograph showing cross sections of the injured carotid arteries of a control rat (×400),

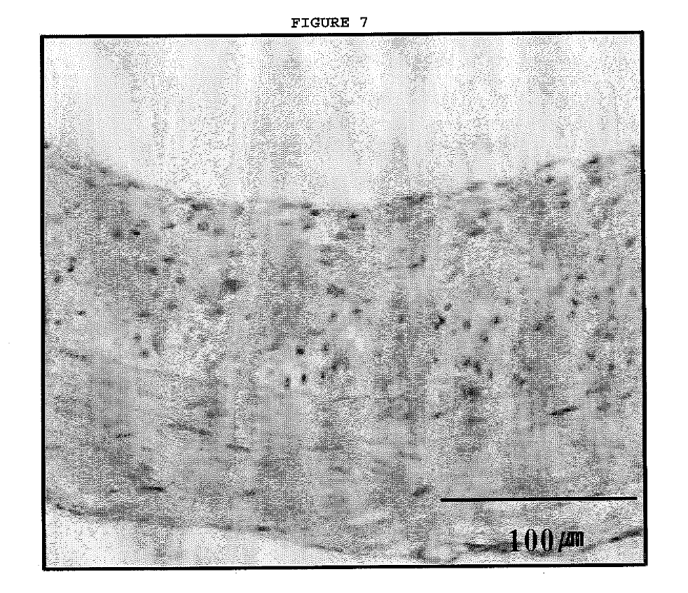

FIG. 8 is photograph showing cross sections of the injured carotid arteries of an obovatol-treated group (×400).

FIG. 9 is a graph showing the inhibitory effect of obovatol on the platelet-derived growth factor-induced hyperplasia of the arterial smooth muscle cells in a dose-dependent over time,

FIG. 10 is a graph showing the inhibitory effect of obovatol on DNA synthesis in arterial smooth muscle cells treated with platelet-derived growth factor.

### DESCRIPTION OF THE PREFERRED EMBODIMENTS

Below, a detailed description will be given of the present invention.

Obovatol can be prepared from Magnolia obovata. For this, leaves of Magnolia obovata are dried in a shady place, sliced, and added to 2 to 20 volumes of a non-polar solvent, such as hexane, chloroform, ethyl acetate, acetone, etc., or a mixture of 1:1.0 to 1:10 of water and a non-polar solvent, and preferably to 2 to 20 volumes of chloroform, followed by extraction at 25° C. for 24 hours. The extraction can be conducted by cold precipitation, reflux condensation, or ultrasonication, and is preferably conducted by cold precipitation. The resulting extract, which is soluble in the non-polar solvent, is fractionated and washed many times with distilled water and purified, optionally followed by typical fractionation (Harborne J. B. Phytochemical methods: A guide to modern techniques of plant analysis., 3rd Ed., pp 6-7, 1998).

For instance, the purified, non-polar solvent extract is concentrated in a vacuum, and the concentrate was fractioned in a mixture of 1:1 ethylacetate:water. The organic layer thus formed is concentrated and the residue is purified by silica gel column chromatography using a mixture of chloroform and methanol and eluted with an elution solvent of various ratios (9:1-6:4) of chloroform and methanol. The resulting eluate was purified by C18 column chromatography, preferably using a solvent mixture of 4:1 methanol:water, and then by HPLC on a Phenomenex Ultracarb 10 ODS column (250×21.2 mm) using an elution solvent mixture of 4:1 methanol:water to afford obovatol, which is represented by the following Chemical Formula 1.

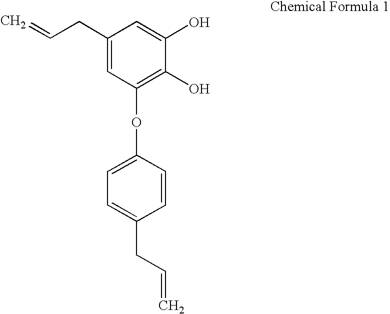

In order to examine whether obovatol can effectively suppress restenosis following a stenting procedure, a histopathological analysis was conducted with a rat carotid artery injury model in which obovatol was applied topically to an injured locus of the exima, showing that neointimal hyperplasia was prevented in obovatol-treated groups in contrast to a control group. When it was applied to arterial smooth muscle cells, which play an important role in restenosis, obovatol was observed to inhibit the proliferation of vascular smooth muscle cells in a dose-dependent manner. It was also observed that obovatol inhibited DNA synthesis in arterial smooth muscle cells in a dose-dependent manner and increased the proportion of cells in a resting state (G₀/G₁) in the cell cycle. Therefore, obovatol is proven to inhibit vascular smooth muscle cell proliferation following stenting procedure, thereby being able to prevent and treat restenosis effectively.

Leaves of Magnolia obovata have been used as a diet or for their therapeutic or medicinal value. Accordingly, extracts or compounds from the leaves are not toxic and cause no side effects.

In accordance with the present invention, there is provided a pharmaceutical composition for the prevention and treatment of restenosis, comprising the obovatol compound isolated from the leaves of Magnolia obovata.

For use in the prevention and treatment of restenosis following a stenting procedure, the pharmaceutical composition according to the present invention comprises obovatol as an active ingredient in combination with a pharmaceutically acceptable carrier.

Obovatol, serving as an active ingredient, may usually be formulated in combination with various pharmaceutically acceptable carriers or excipients into tablets, capsules, soft capsules, liquids, ointments, or injections. Examples of the pharmaceutically acceptable carriers or excipients useful in the present invention include binders (e.g., polyvinylpyrrolidone, hydroxypropylcellulose), disintegrants (e.g., calcium carboxymethyl cellulose, sodium starch glycolate), diluents (e.g., corn starch, lactose, bean oil, crystalline cellulose, mannitol), lubricants (e.g., magnesium stearate, talc), sweeteners (e.g. white sugar, sucrose, sorbitol, aspartame), stabilizers (e.g., sodium carboxymethyl cellulose, alpha or beta cyclodextrin, vitamin C, citric acid, beeswax), preservatives (e.g. paraoxybenzoic acid methyl, paraoxybenzoic acid propyl, sodium benzoate) and/or flavorings (e.g. ethyl vanillin, masking flavor, menthol flavono, herb flavor, etc.)

Ovobatol may be administered at a dosage of 0.0001 to 100 mg per kg of body weight in one dose or in two or three doses a day, depending on various factors including patient's age, sex and symptom, administration route, and administration purpose. It will be apparent to those skilled in the art that the suitable total daily dose may be determined by an attending physician within the scope of sound medical judgment. The specific therapeutically effective dosage level for any particular patient may vary depending on a variety of factors, including the kind and degree of a desired reaction, the specific composition, including the use of any other agents according to the intended use, the patient's age, weight, general state of health, gender, and diet, the time of administration, route of administration, and rate of excretion of the composition; the duration of the treatment; other drugs used in combination or coincidentally with the specific composition; and other factors well known in the medical arts.

The pharmaceutical composition in accordance with the present invention can prevent or treat the restenosis following vascular injury-accompanied procedures including stenting, without side effects, and thus can find various applications in the treatment of coronary arterial diseases.

A better understanding of the present invention may be obtained through the following examples, which are set forth to illustrate, but are not to be construed as the limit of the present invention.

### EXAMPLE 1

### Isolation and Purification of Obovatol

1-1. Preparation of Extract from Leaf of Magnolia obovata

Leaves of Magnolia obovata were collected, dried in a dark place, and finely sectioned. To 3 kg of the sectioned leaves was added 20 liters of a mixture solvent of 1:1 chloroform:acetone, followed by extraction at 25° C. for about 24 hours in a water bath using a reflux condenser. A pool of the resulting extracts was concentrated in a vacuum to afford 200 g of the non-polar solvent extract.

1-2. Isolation and Purification of the Final Compound

200 g of the non-polar solvent extract obtained from leaves of Magnolia obovata in Example 1-1 was fractioned into an aqueous layer and an organic layer. The aqueous layer was washed three times with 1 liter of ethyl acetate and the organic fractions were pooled, along with the organic layer The resulting organic pool was concentrated in a vacuum to form 180 g of a concentrate. This concentrate was dissolved in 500 ml of methanol and adsorbed into 500 g of C18, followed by elution with 1 liter of a solvent mixture of 4:1 methanol:water to give an active fraction. After this active fraction was concentrated in a vacuum, 100 of the concentrate was dissolved in methylene chloride. This solution was loaded onto a column (4.5×40 cm) filled with 1 kg of silica gel (Merck 9385 Silica Gel) along with a solvent mixture of 9:1 hexane:ethyl acetate, followed by two rounds of silica gel column chromatography using a solvent mixture of hexane and ethyl acetate (ratio varying from 9:1 to 6:4) as an eluent. Further purification of the active eluent through HPLC yielded 1 g of an obovatol compound showing the following physical properties.

Obovatol

Empirical Formula: C₁₈H₁₈O₃

Mass: M⁺=282

¹H-NMR (400 MHz, CDC1₃) d ppm: 6.28(H-4, d, J=1.8 Hz), 6.56 (H-6, d, J=1.8 Hz), 3.18 (H-7, d, J=6.61 Hz), 5.97(H-8 and H-8′, m), 5.09 (H-9 and H-9′, m), 6.93 (H-2′ and H-6′, d, J=4.3 Hz), 7.14 (H-3′ and 5′, d, J=4.3 Hz), 3.36 (H-7′, d, J=6.6 Hz); ¹³C-NMR(100 MHz, CDC1₃) d ppm: 143 (C-1)132.93 (C-2), 144.77 (C-3), 110.68 (C-4), 132.47 (C-5), 11.17(C-6), 39.60 (C-7), 137.33(C-8), 115.85(C-9), 154.98 (C-1′), 117.84 (C-2′ and 6′), 129.82 (C-3′ and 5′), 135.18(C-4′), 39.38 (C-7′), 137.18 (C-8′), 115.75 (C-9′).

In the following Examples, values are expressed as mean±standard errors (mean±S.E.). For a significance test of data, an unpaired Student's T-test was used. Each test was independently conducted at least three times. Values of p<0.05 were considered statistically significant.

### EXAMPLE 2

### Test of Obovatol for Prevention of Restenosis in Injured Carotid Artery

(Step 1). Preparation of Obovatol-Containing Pluronic Gel

For the topical application of obovatol in vivo, pluronic gel was employed. F-127 pluronic gel (Sigma Chemical Company, Germany) was dissolved in cold, deionized water to give 40% gel one day before the experiment, and was allowed to stand at 4° C. for 12 hours to dissolve powdered F-127 pluronic gel completely. On the day of the experiment, obovatol was dissolved in a concentration of 10 μg/μL in 100% ethanol and 10 μL of the obovatol solution (10 μg/μL) was mixed with 90 μL of the 40% F-127 pluronic gel to afford 100 μL of obovatol-pluronic gel, comprising 100 μg of obovatol. A control was prepared in the same manner except that no obovatol was contained therein.

(Step 2) Surgery for Carotid Artery Injury

After rats were anaesthetized by abdominal injection with ketamine (50 mg/kg) and xylazine (6.7 mg/kg) and incised to expose the common carotid artery, the external carotid artery and the internal carotid artery were exteriorized through a ventral right line neck incision. While blood flow was temporarily halted by occluding the artery with microvascular clamps (Acland, S&T, Switzerland) at the proximal region of the common carotid artery and the distal region of the internal carotid artery, arteriotomy was performed on the external carotid artery. A 2F Fogarty arterial embolectomy catheter (Baxter Healthcare Corporation, USA) was inserted into the lumen of the right common carotid artery through the incised region and the balloon was inflated to a size larger than the diameter of the common carotid artery so as to generate slight arterial wall resistance. The catheter was advanced a predetermined distance and then withdrawn. This procedure was repeated a total of three times to induce endothelial denudation, after which the catheter (the balloon?) was removed from the arterial lumen. Then, the microvascular clamps were removed to allow blood to flow through the artery. Secretions and blood were completely removed from the exterior of the carotid artery before the obovatol-pluronic gel or the obovatol-lacking pluronic gel were topically applied in an amount of 100 μL to upper loci of the carotid artery, followed by ligation of the artery with a suture. Two weeks after the arteriotomy, a carotid artery angiography was conducted and a carotid artery sample was taken and analyzed histopathologically.

(Step 3) Carotid Artery Angiography

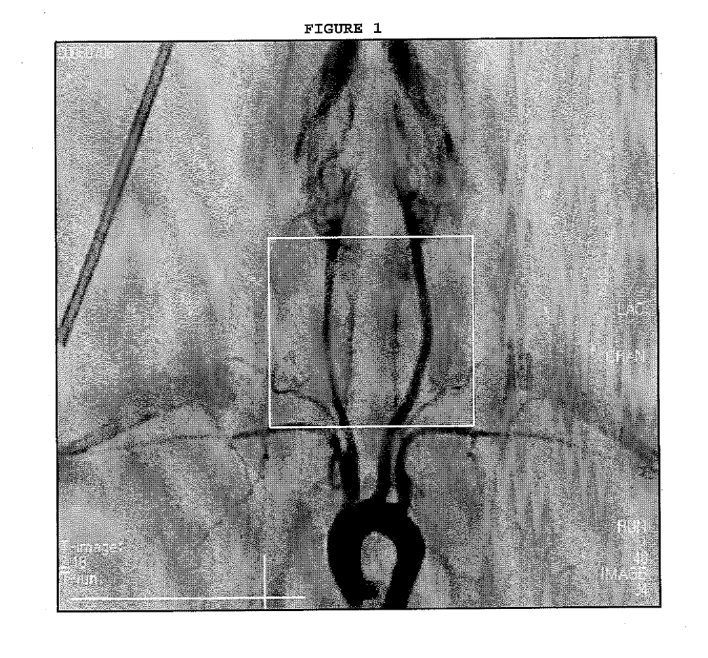

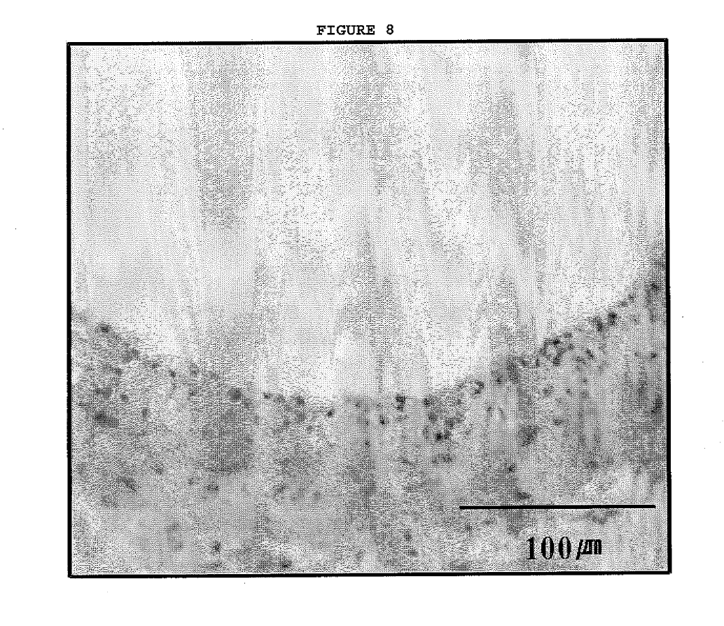

14 days after the arteriotomy, the rats were put under general anesthesia by abdominal injection with ketamine (50 mg/kg) and xylazine (6.7 mg/kg), followed by ventral midline incision. A 4F vascular cannula (Cook, USA) was inserted into the ventral aorta and the catheter was advanced toward the head to a locus as close as possible to a branch between the common carotid arteryand the transverse aorta. After the injection of a contrast media (Visipaque™, Amersham Health, Cork, Ireland), mean luminal diameters (MLD) were measured using computerized coronary angiography (DCI Videodensitometry, Phillips, Netherlands)(See FIG. 1˜FIG. 8).

Carotid artery angiography was also conducted in the rats which did not undergo the arteriotomy. They were measured for the inner diameter of the carotid artery using a 5F coronary catheter, and the mean value thereof was used as a control.

The inner diameters were calculated to be 0.63±0.12 mm for the control and 0.78±0.06 mm (p<0.01) for an obovatol-treated group, which indicated that treatment with obovatol prevented restenosis.

### EXAMPLE 3

### Histopathological Test

The control and the obovatol-treated group were analyzed for intimal thickness, medial thickness, intimal area, intimal-medial area ratio and restenosis degree (%) and the results are summarized in Table 1.

TABLE 1

| | | Obovatol | |
|---|---|---|---|
| | Control | Treated | P Values |
| Intimal | 0.18 ± 0.06 | 0.23 ± 0.02 | P < 0.05 (0.011) |
| Thickness (mm) | | | |
| Medial Area (mm²) | 0.12 ± 0.02 | 0.11 ± 0.10 | P > 0.05 (0.669) |
| Intimal Area | 0.18 ± 0.06 | 0.13 ± 0.03 | P < 0.05 (0.022) |
| (mm²) | | | |
| Intimal-Medial | 1.54 ± 0.48 | 1.12 ± 0.23 | P < 0.05 (0.011) |
| Area Ratio | | | |
| Stenosis (%) | 50.10 ± 13.25 | 34.94 ± 7.23 | P < 0.01 (0.0017) |

As seen in Table 1, similarity was found in medial thicknesses between the obovatol-treated group and the control, indicating that there was no cytotoxicity in obovatol. There is a significant difference in medial thickness between the obovatol-treated group and the control (p<0.05) while a significant decrease in intimal area was found in the obovatol-treated group, compared to the control group (p<0.05). As for the intimal-medial area ratio and the stenosis degree, statistically significant differences were found between the obovatol-treated group and the control group (p<0.05, p<0.01, respectively).

### EXAMPLE 4

### Inhibitory Effect of Obovatol on Arterial Smooth Muscle Cell Proliferation

Obovatol was examined for inhibitory activity against the proliferation of arterial smooth muscle cells in rats. Arterial smooth muscle cells were plated at a density of 3.0×10⁴ cells/well onto 12-well plates containing a DMEM medium supplemented with 0.5% (V/V) fetal bovine serum, and incubated for 24 hours After the addition of obovatol (1, 3, and 5 μM) to the plates, the cells were incubated for 24 hours. Treatment with 50 ng/ml of platelet-derived growth factor (PDGF-BB, Sigma Chem. Co., USA) was followed by incubation for 24, 48, and 72 hours. Thereafter, the cells were trypsinized and counted using a cell counter.

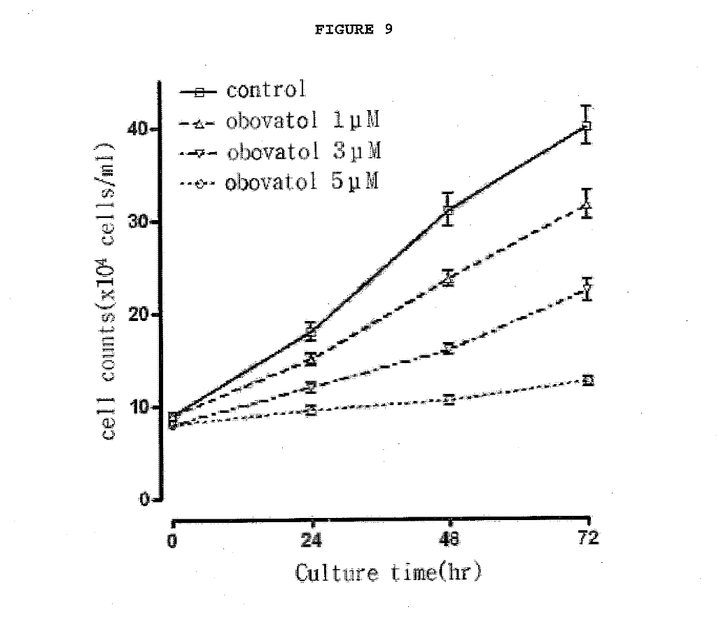

The results are graphed in FIG. 9, in which cell counts are plotted against culture time periods according to the concentrations of obovatol, proving that obovatol is inhibitory of the growth of arterial smooth muscle cells.

### EXAMPLE 5

### Inhibitory Effect of Obovatol on DNA Synthesis in Arterial Smooth Muscle Cells

This experiment was undertaken to examine whether the inhibitory effect of obovatol on cell proliferation shown in Example 4 was attributed to the inhibition of DNA synthesis.

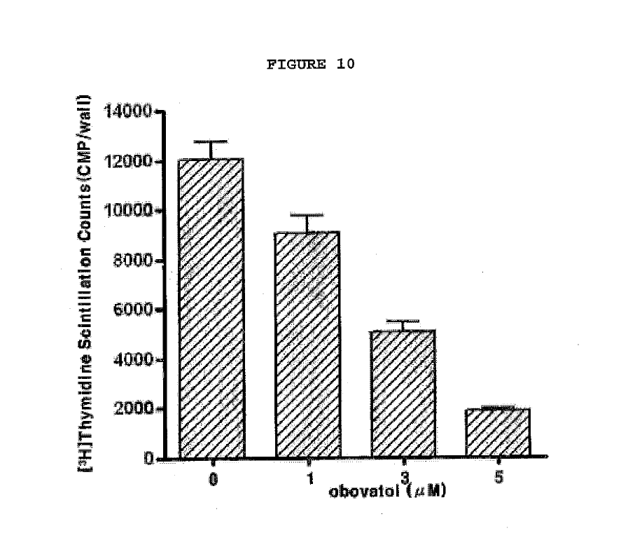

Arterial smooth muscle cells were cultured in the same manner as in Example 4, with the exception that the cells were cultured for 20 hours after treatment with platelet-derived growth factor, and then cultured for 4 hours in the presence of 1 μl of [3H] thymidine. The cultured cells were washed with PBS and incubated with 500 μl of TCA (trichloroacetic acid) for 30 min. After the removal of TCA, the cells were washed with a mixture of 1:1 ethanol:ether (v/v) and disrupted with 500 μl of NaOH. The cell lysate was mixed with 5 ml of a scintillation cocktail and measured for radioactivity to analyze relative amounts of newly synthesized DNA. FIG. 10 is a graph in which amounts of newly synthesized DNA in PDGF-treated arterial smooth muscle cells are plotted against concentrations of obovatol. As seen in the graph, the DNA synthesis of the arterial smooth muscle cells decreases with the increasing concentration of obovatol.

Therefore, the inhibitory effect of obovatol on arterial smooth muscle cell proliferation is attributed to the fact that obovatol suppresses DNA synthesis therein.

### EXAMPLE 6

### Acute Toxicity Assay

ICR mice (weighing 25±5 g) and SPF Sprague-Dawley rats (weighing 235±10 g) were separately divided into four groups of three and abdominally injected with the obovatol prepared in Example 2 at doses of 1000 mg/kg, 100 mg/kg, and 10 mg/kg and monitored for toxicity over 24 hours.

Death was observed in none of the four groups, with no abnormality observed in any of the animals, such as in body weight, food intake, etc., compared to the control. Therefore, the compound of the present invention was proven to be safe to the body.

### EXAMPLE 7

### Formulation of Pharmaceutical Compositions for Prevention and Treatment of Restenosis

Obovatol in accordance with the present invention was formulated in combination with auxiliary agents, such as excipients, binders, lubricants, disintegrants, diluents, etc., into pharmaceutical preparations as follows.

### PREPARATION EXAMPLE 1

### Tablet

Using a conventional tabletting process, 10 mg of obovatol, 20 mg of lactose, 20 mg of starch and a suitable amount of magnesium stearate were formulated into a 50 mg tablet useful in the prevention and treatment of restenosis.

### PREPARATION EXAMPLE 2

### Capsule

Using a conventional process, 10 mg of obovatol, 20 mg of lactose, 19 mg of starch, 1 mg of talc and a suitable amount of magnesium stearate were loaded into a capsule to afford a capsule medicine useful in the prevention and treatment of restenosis.

### PREPARATION EXAMPLE 3

### Liquid

According to a typical process, 100 mg of obovatol, 10 g of isomerized sugar, 500 mg of honey, 20 mg of nicotinic acid amide (pharmacopoeia), 30 mg of anhydrous caffeine (pharmacopoeia) 30 mg, and 70 mg of sodium benzoate were formulated and loaded in a 100 ml brown container which was then tightly sealed and pasteurized to afford a liquid preparation useful in the prevention and treatment of restenosis.

### PREPARATION EXAMPLE 4

### Injection

According to a typical injection preparation process, 6 mg of obovatol was formulated in combination with a suitable amount of sterile water and loaded in a 2 ml ampule which was then tightly sealed and sterilized to afford an injection useful in the prevention and treatment of restenosis.

### PREPARATION EXAMPLE 5

### Soft Capsule

According to a typical process, 10 mg of obovatol, 230 mg of polyethylene glycol and 13 mg of glycerin were loaded into an envelope made of 52 wt % of gelatin, 32 wt % of glycerin, 12 wt % of ANIDRISORB 35/70 and 5 wt % of water to afford a soft capsule medicine useful in the prevention and treatment of restenosis.

### PREPARATION EXAMPLE 6

### Granules

Using a typical granulation extruder, 10 mg of obovatol and 25 mg of lactose were formulated into granules useful in the prevention and treatment of restenosis.

As explained and proven hitherto, a pharmaceutical composition comprising obovatol as an active ingredient is provided for the prevention and treatment of restenosis following a stenting procedure, in accordance with the present invention. Being useful in the prevention of restenosis following a blood injury procedure including a stent, the pharmaceutical composition of the present invention is applicable to the treatment of various vascular coronary artery diseases.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A pharmaceutical composition for the prevention and treatment of restenosis following a blood vessel injury procedure, comprising obovatol, represented by the following Chemical Formula 1, as an active ingredient:

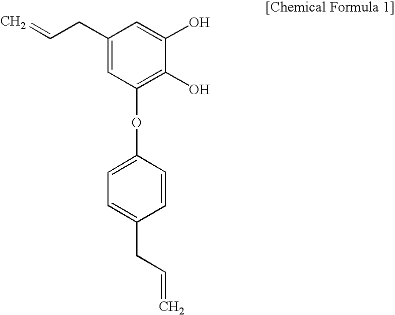

2. The pharmaceutical composition according to claim 1,
wherein the pharmaceutical composition is in a dosage form selected from a group consisting of a capsule, a liquid, an injection, a soft capsule, a granule and a tablet.

3. The pharmaceutical composition according to claim 1,
wherein the blood vessel injury procedure is percutaneous transluminal coronary angioplasty, balloon angioplasty, stent insertion, coronary artery bypass graft surgery, or arteriovenous anastomosis.

4. The pharmaceutical composition according to claim 1, wherein the pharmaceutical composition is administered at a dosage of 0.0001˜100 mg per kg of weight in one dose or in two or three doses a day.
